(19) United States
(12) Patent Application Publication
(10) Pub. No.: US 2005/0191363 A1
(43) Pub. Date: Sep. 1, 2005

(54) Method for inhibiting fibrogenesis by a mixture of natural peptides from porcine liver extract

(76) Inventors: Mengsu Yang, Hong Kong (CN); Hsiang-fu Kung, Hong Kong (CN); Yaou Zhang, Shenzhen (CN); Celina S.M. Cheng, Hong Kong (CN); Pik Yuen Cheung, Hong Kong (CN)

Correspondence Address: Robert K. Tendler, 65 Atlantic Avenue, Boston, MA, 02110, US

(21) Appl. No.: 10/884,782

(22) Filed: Jul. 2, 2004

Related U.S. Application Data
(60) Provisional application No. 60/484,680, filed on Jul. 2, 2003.

(51) Int. Cl. A61K 35/407

(52) U.S. Cl. 424/553

(57) ABSTRACT

The present invention relates to a method for inhibiting hepatic fibrogenesis, especially liver fibrogenesis, which method comprises administering an effective amount of Fibroscutum, a mixture of natural peptides extracted from the liver tissue of suckling pig, containing at least 6 peptides of relative abundance of 10-27% and molecular weight of 7-40 kD.

### CROSS-REFERENCE TO RELATED APPLICATIONS

This Application claims rights under 35 USC 119(e) from U.S. Application 60/484,680 filed on Jul. 2, 2003, the contents of which are included herein by reference.

### FIELD OF THE INVENTION

This invention relates to a method for inhibiting fibrogenesis, especially liver fibrogenesis, by administering an antagonist of hepatic stellate cell activation and fibroblast cell activation.

### BACKGROUND OF THE INVENTION

### Liver Fibrogenesis

Fibrosis has an integral role in the final common pathway of structural remodeling that reduces normal organ function following injury. It is one of the most fundamentally destructive and unwanted responses to developmental or inflammatory diseases and is seen in millions of individuals in the advanced stages of many different disease processes including such diseases as cystic fibrosis, interstitial nephritis, hepatic cirrhosis and pulmonary fibrosis following exposure to high oxygen tension. Liver fibrosis is characterized by an excessive deposition of extracellular matrix components in the liver. Several liver cell types participate in matrix deposition, the major types being hepatic stellate cells (HSC) (Friedman et al., 2000) and portal fibroblasts (Tuchweber et al., 1996). During the past decade, a lot of attention has been given to the stimuli responsible for fibrogenic cell activation in the liver. The major focus has been on growth factors and oxidant stress (Friedman et al., 2000).

Fibrogenesis is classically mediated by organ fibroblasts that express abundant amounts of collagen types I and III. The expression of fibrogenesis in liver has been the subject of intense study over the last several years. The cytokine regulation of this process is complex. It is generally believed that collagen types I and III are the principal fibrotic collagens, and that they are well expressed by hepatic stellate cells (HSC) and fibroblast cells. The expression of these collagens is regulated by a complexity of cytokines. Tumor necrosis factor beta 1 (TGF.beta.1), for example, is an early and pivotal component in the stimulatory process. It is generally believed that TGF.beta.1 appears to be a regulatory molecule for these collagen genes in the lungs, liver, and kidney. (Wahl, S. M., 1992 J. Clin. Immunol. 12:61-74; Sharma, K. and Ziyadeh, F. N., 1993 Seminars in Nephrology 13:116-128; Roberts et al., 1986 Proc. Nat'l Acad. Sci. USA 83:4167-4171.)

The most important causes of liver fibrosis cirrhosis are chronic hepatitis B and C infection, and prolonged alcohol abuse. Liver cirrhosis is the clinical end point of liver fibrosis. Until present time, effective methods for reversing treatment of liver cirrhosis are not available, and those with life threatening impairment of liver function can only look to liver transplants for salvage. However, each year the number of new cases of cirrhosis exceeds the number of livers available for transplantation by a factor of 5 to 10. Therefore, prevention of fibrogenesis and early treatment of fibrosis is the best treatment of cirrhosis (Achord J L. 1991, Compr Ther. 17:57-64, Habib etal., 2001, Postgrad Med. 109:101-13). The strategies of inhibiting fibrogenesis can be grouped as: (a) Anti-inflammatory agents and antioxidants; (b) Antagonists of cytokines or cytokine receptors; (c) Inhibitors of stellate cell activation; and (d) Anti-collagen agents (D. Montgomery Bissell., 2001, EXPERIMENTAL and MOLECULAR MEDICINE, Vol. 33:179-190). However, most of these agents are either not very effective in treating fibrosis or having severe side effects.

There is thus a need to develop new methods for inhibiting liver fibrogenesis and treating liver fibrosis.

### Hepatocyte Promoting Factors and Wei Jia

Fulminant hepatic failure in acute hepatitis is caused by massive hepatocyte necrosis and cirrhosis resulting from prolonged, widespread hepatocyte necrosis and apoptosis. Therefore, agents that can protect hepatocytes from necrosis and apoptosis and stimulate liver cells growth and repair are screened for the treatment of severe hepatitis and cirrhosis. Hepatocyte promoting factors including hepatocyte growth factor (HGF), HGF activator, hepatic stimulator substance (HSS), hepatocyte growth promoting factor (hepatopoietin), and augmenter of liver regeneration (ALR) are the candidates of such agents. HGF has been studied extensively and shown to be a potent mitogen for mature parenchymal hepatocyte cells with anti-apoptotic effects in vivo and it can effectively prevent endotoxin-induced fulminant hepatic failure in mice. However, HGF also acts as an endothelial cell specific growth factor and an angiogenic growth factor for a broad spectrum of tissues and cell types (including tumors), which may cause severe side effects such as enhancing tumor invasion. Hence, it is very important to search for new hepatocyte promoting factors that have high therapeutic effectiveness and low side effects.

Wei Jia is a Category I new drug of Sinogen Pharmaceuticals of Weihai, Shandong, a subsidiary of LifeTec Enterprise Ltd. The drug has biological activities of hepatocyte promoting factors, but it is not one of the factors mentioned above. Table 1 lists the hepatocyte promoting factors and their molecular weights. Note, Wei Jia is a distinct hepatic peptide factor.

TABLE 1
The hepatocyte promoting factors and their molecular weights.

| Name | Molecular weights | |
|---|---|---|
| HGF | 82 | kDa |
| hepatopoietin | 10.7 | kDa |
| HGF activator | 34 | kDa |
| Hepatic stimulator substance | 12.4-17.5 | kDa |
| ALR | 15 | kDa |
| Wei Jia | 21 | kDa |

Historically, Wei Jia is developed based on more than a decade of research in China. As early as 1987, researchers at Hunan Medical University isolated a hepatic peptide factor (HPF) from the liver tissue of suckling pig, which was active in stimulating the DNA synthesis of hepatocytes. Using ion-exchange chromatography and HPLC, it was found that the HPF composed essentially three fractions of protein. The first and the second were minor fractions of MW>30 kDa which had no biological activity, while the third was a major fraction (60.4% of the total protein) of MW 21 kDa with marked biological activity. This peptide was later purified to a single band in SDS-PAGE and was termed “Hepatocyte DNA Synthesis Stimulating Factor (HDSSF)”.

After extensive pre-clinical studies on HDSSF, including acute and long-term toxicity, mutagenesis and carcinogenesis, reproductive toxicity, drug dependence, pharmacodynamics and pharmacokinects, Sinogen acquired the right to develop the drug and filed for IND (investigative new drug) in SDA of China with the brand name Wei Jia. Three phases of clinical trials were conducted under the coordination of SDA, with 691 patients from 42 hospitals across 6 different regions of China participating in the last phase of clinical studies. The consolidated results of phase III clinical trials showed that the efficiency of Wei Jia in combination therapy for the treatment of severe chronic and acute hepatitis was as high as 88.9% and 78.4% respectively. Based on these results, Wei Jia was granted by SDA of China Category I new drug status and approved for sale in 2001 with 12 years of license protection.

### Fibroscutum

Recently, the production procedure of Wei Jia was modified and a new product was developed which shows different components and biological activity. A new name was applied to the new product, namely Fibroscutum.

As will be described hereinafter, Fibroscutum is a mixture of peptides extracted from the liver tissue of suckling pig, and as such is a porcine liver extract. Fibroscutum contains 13-17 components, 6-7 of them are major components including, HMG-17; Decorin; Prolactin receptor, Hepatoma-derived growth factor-related protein 1 or Thymic peptide; Nucleolar protein Tsg118 or Ubiquitin. Since diseases such as cancer result from uncontrolled cell proliferation and fibrotic disorders related to over proliferation of fibroblasts, the subject invention can be used to treat cancer and fibrotic disorders, and the group consisting of glomerulonephritis, ARDs, cirrhosis of the livers, fibrotic cancer, fibrosis of the lungs, post myocardial infarction, cardiac fibrosis, post-angioplasty restinosis, renal interstitial fibrosis, and scarring or a diabetes-associated pathology.

The present invention is also directed to a process for the production of Fibroscutum. In the process, livers from piglets are homogenized and mixed at 500 rpm for 1 hr at room temperature, then, heated at 95° C. After filtration and cooling, the sample is purified with DEAE-52 Sepharose using a fast flow technique. Finally, the samples passed pre-product test and are sterilized by filtration with a 0.2 u filter and then bottled.

### SUMMARY OF THE INVENTION

It has been found that Fibroscutum is an antagonist that attenuates liver fibrosis by inhibiting liver fibrogenesis.

This conclusion is the result of investigating the effects of antagonism on fibrotic conditions related to TGF.beta.1 induced hepatic stellate cell activation and TGF.beta.1 induced fibroblast cell activation as it relates to liver fibrosis. Specifically, the effect of Fibroscutum on hepatic stellate cell activation and fibroblast cell activation was studied. The subject invention thus provides a method for inhibiting fibrogenesis, especially liver fibrogenesis that involves administering an effective amount of a hepatic stellate cell activation and fibroblast cell activation antagonist to a patient in need of such treatment.

In one embodiment, the antagonist is Fibroscutum, which prevents the development of liver fibrosis, especially in course of a viral hepatitis, such as chronic hepatitis B and chronic hepatitis C.

Fibroscutum is a mixture of natural peptides extracted from the liver tissue of suckling pig, containing at least 6 peptides of relative abundance of 10-27% and molecular weight of 740 kD. More particularly, Fibroscutum is a porcine liver extract. Fibroscutum contains at least six components including, HMG-17; Decorin; Prolactin receptor, Hepatoma-derived growth factor-related protein 1 or Thymic peptide; Nucleolar protein Tsg118 or Ubiquitin.

It has been found that Fibroscutum exhibits inhibitory effects on cell proliferation of fibroblast. Since diseases such as cancer result from uncontrolled cell proliferation and fibrotic disorders related to over proliferation of fibroblasts, the subject invention can be used to treat cancer and fibrotic disorders, and the group consisting of glomerulonephritis, ARDs, fibrosis and cirrhosis of the livers, fibrotic cancer, fibrosis of the lungs, post myocardial infarction, cardiac fibrosis, post-angioplasty restinosis, renal interstitial fibrosis, and scarring or a diabetes-associated pathology.

The present invention is also directed to a process for the production of Fibroscutum. In the process, first livers from piglets are homogenized and are then mixed at 500 rpm for 1 hr at room temperature and heated at 95° C. After filtration and cooling, the sample is purified with DEAE-52 Sepharose using a fast flow technique. Finally, the samples that have passed pre-product test are sterilized by filtration with a 0.2 u filter and then bottled.

It is noted that Wei Jia, mentioned above and Fibroscutum are different. Wei Jia and Fibroscutum are quite different in three aspects due to the alteration in the production procedure used to make the substance. First, Fibroscutum has different components as compared to Wei Jia. Fibroscutum consists of six major components in SDS PAGE stained with Coomessie Blue. The molecular weight of the six peptide fractions are about 40, 25, 21, 14-12, 10, and 7 kDa, and their relative ratio of protein are 11, 18, 10, 27, 19 and 15%. Wei Jia only has three components. The molecular weight of the first and the second peptide fraction of Wei Jia is over 30 kDa, and the third one was a major fraction of Wei Jia, having a relative ratio of 60.43% of the total protein. Note, the molecular weight of Wei Jia is 21 kDa.

Second, Wei Jia and Fibroscutum show different biological activities. It was found that Wei Jia injections could effectively promote the hepatocyte to synthesize DNA, both in vitro and in vivo, while Fibroscutum could not stimulate cell proliferation, but rather inhibited tumor cell growth. Fibroscutum also inhibits the growth of fibroblasts stimulated by TGF beta. In clinic, Wei Jia is used to treat hepatitis, while Fibroscutum is designed to treat tumor, especially liver tumor and to prevent fibrotic conditions, especially cirrhosis of the livers.

Finally, the known sequences of the major components in Wei Jia and in Fibroscutum are different. In Wei Jia, only the 21 kDa major peptide was sequenced and the first 10 amino acids were identified as DEKSFKWKTA. After blasting, it was found that the sequence of the 10 amino acids was matched to transaldolase, a 336-amino acid protein. It is the key enzyme of the pentose phosphate pathway. Among 6 major peptides of Fibroscutum, 5 of them were sequenced, Table 2 shows the results and none of these peptides are transaldolase.

TABLE 2
The result of N-terminal sequence of major Fibroscutum peptides

| | | | | |
|---|---|---|---|---|
| | | sequence of amino | sequence of amino | |
| samples | MW(kDa) | acid (1) | acid (2) | Protein Identification |

| | | | | |
|---|---|---|---|---|
| 1 | 39.810 | blocked | (SGVDI) | Calponin 2 |
| 2 | 24.923 | DEAAGIGP | DEAAGIAP | Decorin |
| 3 | 20650 | KAPAKK | SAPAKK | Non-histone chromosomal |
| | | | | protein HMG-17 |
| 4a | 14175 | PEPAKKAPAA | | 1. Prolactin receptor |
| | | | | 2. Thymic peptide |
| | | | | 3. hepatoma-derived |
| | | | | growth factor-related |
| | | | | protein 1 |
| 4b | 12058 | PEPAKKA | | Same as 4a |
| 5 | 10199 | AKKRKKK | AVKRKKK | 1. Nucleolar protein |
| | | | | 1 Tsg118 |
| | | | | 2. Ubiquitin |
| 6 | 7339 | AKKRKKK | | Same as 5-2 |

### BRIEF DESCRIPTION OF THE DRAWINGS

These and other features of the subject invention will be better understood in connection with a Detailed Description, in conjunction with the Drawings, of which:

FIGS. 1A and 1B illustrate the effect of Fibroscutum treatment on liver fibrosis after CCl.sub.4 (carbon tetrachloride) treatment (Sirius red staining), with FIG. 1A showing the results of a rat treated with CCl.sub.4 alone for eight weeks and FIG. 1B showing a rat treated with CCl.sub.4 alone for two weeks and both CCl.sub.4 and Fibroscutum for six weeks. (magnification: ×100);

FIG. 2 is a graph showing the effect of Fibroscutum on hepatic stellate cell proliferation induced by TGF.beta.1 in which LX-2 cells (Human HSC cell line) were exposed to Fibroscutum, either alone, or in the presence of 2 ng/ml TGF.beta.1 for 48 hours;

FIG. 3 illustrates the effect of Fibroscutum treatment on hepatic MMP-2 (matrix metalloproteinases-2) and TIMP-1 (tissue inhibitor of matrix metalloproteinases-1) mRNA levels of HSC cells stimulated with TGF.beta.1 in which LX-2 cells were treated with Fibroscutum, either alone, or in the presence of 2 ng/ml TGF.beta.1 for 24 hours and in which the cells were harvested for total RNA extraction;

FIG. 4 is a graph illustrating the effect of Fibroscutum treatment on collagen I protein level of HSC cells stimulated with TGF.beta.1 in which LX-2 cells were treated with Fibroscutum, either alone, or in the presence of 2 ng/ml TGF.beta.1 for 24 hours and in which the cells were harvested and lysated to obtain protein samples;

FIG. 5 is a graph illustrating the effect of Fibroscutum treatment on collagen III protein levels of HSC cells stimulated with TGF.beta.1, in which LX-2 cells were treated with Fibroscutum, either alone, or in the presence of 2 ng/ml TGF.beta.1 for 24 hours and in which the cells were harvested and lysated to obtain protein samples;

FIG. 6 is a graph illustrating the effect of Fibroscutum treatment on fibroblast cell proliferation induced by TGF.beta.1 in which NIH 3T3 Cells were exposed to Fibroscutum, either alone, or in the presence of 2 ng/ml TGF.beta.1 for 48 hours, and in which the cell number was evaluated through the measurement of the reduction of the dye 3-(4,5-dimethylthiazol-2yl)-2,5 diphenyltetrazolium (MTT).

FIG. 7 is a flow chart for one embodiment of the production of Fibroscutum; and,

FIG. 8 is a graph illustrating the separated components of the peptide mixture of Fibroscutum by SDS PAGE separation and Coomassie Blue Staining.

### DETAILED DESCRIPTION

### Identification of Major Peptides in Fibroscutum

Fibroscutum is a mixture of peptides extracted from the liver tissue of suckling pig. The peptide mixture can be separated into 6-7 major bands in 17% SDS-PAGE gel stained with Coomessie Blue. As will be described hereinafter, FIG. 8 illustrates the separated components of the peptide mixture of Fibroscutum by SDS PAGE separation and Coomassie Blue staining.

The major peptide components of Fibroscutum have been sequenced and five partial amino acid sequences were obtained. Fibroscutum was separated with SDS PAGE (17%), then transfer to PVDF membrane and stained with Coomassie Blue. Table 2 above shows the result of N-terminal sequence analysis of the peptides.

The sequencing result was obtained using a Procise Amino Acid Sequencer.

The sequence is defined after comparing the sequencing peak profiles and used for blast analysis.

For same samples two sequences were defined for blast. They have similar scores in blast results, but the first one is more reliable.

### Effects of Fibroscutum on Liver Fibrogenesis

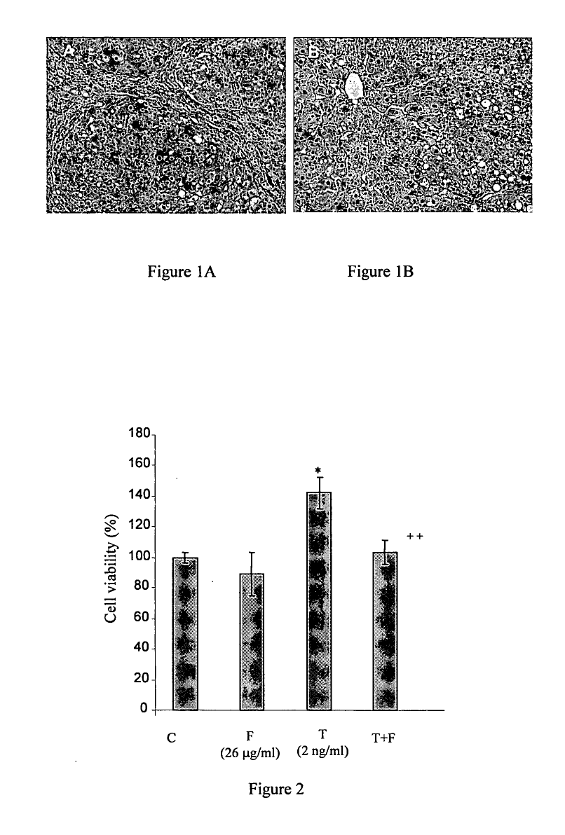

The effect of Fibroscutum on carbon tetrachloride (CCl₄) induced liver fibrosis in rats can be seen in FIG. 1A. Here, Fibroscutum was first investigated. Compared with controls, eight weeks of CCl₄ treatment induced significant fibrosis. This is indicated by the formation of nodules that lack a central vein and the disappearance of the normal structure of hepatic lobules. Two weeks following the initial CCl₄ treatment, Fibroscutum was injected together with CCl₄ for the remaining six weeks. As can be seen in FIG. 1B, addition of Fibroscutum significantly decreased the pathologic changes of fibrogenesis.

Also investigated was the effect of Fibroscutum on hepatic stellate cell (HSC) activation induced by TGF-β. HSC activation is an essential process of liver fibrogenesis. Fibrogenesis presents as proliferation of HSC and fibroblast, and remodeling of extra cellular matrix, including, degradation of collagen IV in the basement membrane of liver cells and the deposition of an excess of extracellular matrix components, such as collagen I and collagen III in the liver. In this process, expression of matrix metalloproteinase 2 (MMP-2) and tissue inhibitor of metalloproteinase 1 (TIMP-1) in mRNA level are up-regulated. Collagen IV is the substrate of MMP-2. TIMP-1 is an inhibitor of MMP-1 which can clear up the deposition of collagen I and III.

It was found that Fibroscutum abrogated the effects of TGF-β on hepatic stellate cells through suppressing cell proliferation, collagen I and collagen III production, and MMP-2 and TIMP-1 expression stimulated by TGF-β.

Also investigated was the effect of Fibroscutum on fibroblast cell proliferation induced by TGF-β. Fibrogenesis also presents as proliferation of fibroblast, and fibrotic disorders are related to over proliferation of fibroblasts. It was found that Fibroscutum also inhibited proliferation of fibroblast induced by TGF-β.

In light of these results and taking into account the fact that Fibroscutum has a very good safety profile, Fibroscutum can be used as a drug against fibrogenesis, especially liver fibrogenesis.

Liver fibrogenesis is the active process leading to the deposition of an excess of extracellular matrix components in the liver. It is observed in a number of conditions such as chronic viral hepatitis B and C, alcoholic liver disease, drug-induced liver disease, hemochromatosis, auto-immune hepatitis, Wilson disease, primary biliary cirrhosis, sclerosing cholangitis, liver schistosomiasis and others. Fibrogenesis can occur similarly in other organs, such as lung, kidney, pancreas, heart and skin.

The subject invention is particularly helpful in the treatment of liver fibrosis. Liver fibrosis is the established excess deposit of extracellular matrix components in the liver. Its endpoint is liver cirrhosis.

In a preferred aspect of the invention, Fibroscutum is useful to prevent the development of liver fibrosis that may occur in a patient infected by Hepatitis virus, e.g. hepatitis B virus (HBV), or hepatitis C (HCV) virus.

Note that chronic viral hepatitis usually involves chronic hepatitis B and chronic hepatitis C.

By way of definition, as used herein the term “patients in need of such treatment” means any human subject or mammals, including sheep, cattle, dogs, cats, rodents, rabbits or goats, who suffer from an organ disease wherein fibrogenesis is observed or generally results from the development of the disease.

The terms “treatment” and “prevention” include therapy and prophylaxis toward fibrogenesis, at any stage of development of the phenomenon or before it occurs.

The invention is especially directed at preventing, or reducing or alleviating liver fibrosis in patients suffering from an organ disease.

The use of therapeutically effective amounts of Fibroscutum in accordance with the subject invention effectively reduces or prevents the development of liver fibrosis.

In the context of the present invention, in one embodiment Fibroscutum is a mixture of natural peptides extracted from the liver tissue of suckling pig, containing at least 6 peptides of relative abundance of 10-27% and molecular weight of 7-40 kD.

### Hepatic Stellate Cell (HSC) Activation

In all tissues, activation of resident mesenchymal cells is a key event in the development of fibrosis. In the liver, this mesenchymal cell is represented by the hepatic stellate cell or HSC, otherwise referred to as Ito cells, fat-storing cells, or lipocytes. (Friedman S L. 1993, N Engl J Med. 328(25):1828-35; Friedman S L. 1996, Prog Liver Dis. 14:101-30.) Subsequent to acute or chronic liver damage, HSC undergo activation, a process characterized by the transformation of resting cells into proliferative, fibrogenic, and contractile myofibroblast-like cells. Activated HSC contribute to the tissue repair process, namely the reconstitution of an extracellular matrix (ECM) network necessary for tissue regeneration. In cases of acute/self-limited tissue damage these changes are self-limited and effective. In contrast, cases of persistent liver injury result in chronic inflammation and lead to the accumulation of extracellular matrix. The reasons for the chronicity are not clear, but could reflect the presence of mediators unique to chronic injury, or more likely, failure of compensatory mechanisms, such as down regulators of inflammation, or matrix protease activity, to keep pace with the ongoing fibrosis.

A cascade of events involving soluble stimuli, matrix-related changes, and altered gene expression results in the activation of HSC. Activation consists of early initiation and late perpetuation phases. Early activation appears to be provoked by at least two stimuli, namely rapid deposition of cellular fibronectin, and release of soluble stimuli by Kupffer cells (hepatic macrophages). The late phase of activation consists of at least five discrete phenotypic changes: (1) proliferation, (2) mitogenesis, (3) contractility, (4) release of proinflammatory cytokines, and (5) matrix protease release.

Activation and proliferation of HSC in liver injury is associated with de novo expression of many cytokine receptors, including epidermal growth factor (EGF-R), transforming growth factor (TGF).beta.-R types I, II, and III, endothelial receptor (ET-R), vascular endothelial growth factor (VEGF)-R, thrombin-R and platelet derived growth factor (PDGF)-R (Friedman, S. L., 1997, Journal of Gastroenterology 32:424-430; Ankoma-Sey, V. M. et al., 1998, Oncogene 17:115-121; Friedman, S. L., 1989, Journal of Clinical Investigation 84:1780-1785; Wong, L. G et al., 1994, Journal of Clinical Investigation 94:1563-1569). Moreover, HSC activation is associated with the expression of several cytokines, growth factors and inflammatory mediators, including EGF, FGF, ET-1, insulin-like growth factor (IGF), thrombin, TGF .alpha., TGF .beta., hepatocyte growth factor (HGF), stem cell factor (SCF), and PDGF (Friedman, S. L., 1997, Journal of Gastroenterology 32:424-430). HSC activation is also associated with an increase in the production of extracellular matrix components, namely collagen types I, III, IV, V, VI, XIV, proteoglycans, and glycoproteins, including fibronectin, laminin, and tenascin. Furthermore, HSC activation is associated with the production of matrix proteases, including MMP-2, stromelysin-1 (transin), MMP-1 (interstitial collagenase), and MT-MMP (membrane type-matrix metalloproteinase), and protease inhibitors, including TIMP-1, TIMP-2, and PAI-1. Thus, the expression of several different factors is associated with HSC activation.

As described in detail in the Examples hereinafter, it has been found that Fibroscutum prevents fibrogenesis in vivo in hepatic injury induced by CCl.sub.4. Though a precise understanding of the molecular basis for the prevention of fibrogenesis of Fibroscutum is not necessary to practice the present invention successfully, the experimental results indicate that the prevention is mediated, at least in part, through inhibition of stellate cell activation stimulated by TGF.beta.1. TGF.beta.1 is an early and pivotal component in the stimulatory process and appears to be a regulatory molecule for collagen genes in the lungs, liver, and kidney.

Note that stellate cell activation is a critical step in hepatic fibrosis. Because Fibroscutum prevents hepatic stellate cell activation and proliferation as described in Example 1, the present invention contemplates using the compound in the treatment of hepatic fibrosis. Similarly, Fibroscutum is also effective at preventing and treating hepatic disorders associated with fibrosis.

### Pharmaceutical Compositions

Fibroscutum is formulated as pharmaceutical compositions useful for inhibition of fibrogenesis, especially liver fibrogenesis.

For this purpose, the compounds of the present invention may be administered parenterally (including subcutaneous injections, intravenous, intramuscular, intrasternal injection or infusion techniques), in dosage unit formulations containing conventional non-toxic pharmaceutically-acceptable carriers, adjuvants and vehicles.

Pharmaceutical compositions containing Fibroscutum alone or in combination with another active ingredient may thus be in the form of sterile injectable preparations, for example, as sterile injectable aqueous or oleagenous suspensions or suppositories.

The injectable solutions or suspensions may be formulated conventionally using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono-or diglycerides, and fatty acids, including oleic acid.

When rectally administered in the form of suppositories, these compositions may be prepared by mixing the drug with a suitable non-irritating excipient, such as cocoa butter, synthetic glyceride esters or polyethylene glycols, which are solid at ordinary temperatures, but liquidify and/or dissolve in the rectal cavity to release the drug.

Compounds of this invention may be administered in any of the foregoing compositions and according to established dosage regimens whenever specific inhibition of the human hepatic stellate cell activation and proliferation is required.

The daily dosage of the products may be varied over a wide range from 0.01 to 1,000 mg per adult human per day. A medicament typically contains from about 0.01 mg to about 500 mg of the active ingredient, preferably, from about 1 mg to about 100 mg of active ingredient. An effective amount of the drug is ordinarily supplied at a dosage level of from about 0.0002 mg/kg to about 20 mg/kg of body weight per day. Preferably, the range is from about 0.001 to 10 mg/kg of body weight per day, and especially from about 0.001 mg/kg to 7 mg/kg of body weight per day. The compounds may be administered on a regimen of 1 to 4 times per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As a typical example, Fibroscutum may be administered in the form of intravenous injections, at a daily dose of 0.03 to 1 mg.

Fibroscutum may be used alone at appropriate dosages defined by routine testing in order to obtain optimal antagonism of the hepatic stellate cell activation and proliferation while minimizing any potential toxicity. In addition, co-administration or sequential administration of other agents is efficacious, including agents aimed at either the etiological treatment of the liver disease such as antiviral agents, corticosteroids or others, or other potential antifibrotic drugs including anti-oxidants, pentoxifylline, silymarin and others.

The following Examples are illustrative of the effect of Fibroscutum.

### EXAMPLES

### Example 1

### Inhibition of Rat Liver Fibrogenesis through Fibroscutum

### Material and Methods

### Animals and Experimental Design

All experiments were carried out using accepted ethical guidelines. Male Wistar rats (Beijing Medical University) weighing 200-250 g were used in this study. The animals had free access to food and drinking water. 40% CCl4 (Sigma) was prepared by mixing CCl4 with olive oil. Liver damage and fibrosis was induced through giving 40% CCL4 at 3 ml/kg body weight by subcutaneous injection, two times a week for four weeks. Control animals for CCL4 received only olive oil.

Fibroscutum was intraperitoneally administered daily at the dose of 1.25 ug/kg/d body weight.

Thus, 3 groups including each 7 animals could be distinguished. Group I: olive oil alone; group II: 40% CCL4; group III: 40% CCL4 plus Fibroscutum. Fibroscutum treatment was started at the third week and lasted for three weeks. In another series of experiments, animals were similarly treated by olive oil or 40% CCL4 for 8 weeks. Fibroscutum treatment was also started at the third week but lasted for six weeks. In that case, all groups included 7 animals.

At the designated time points, the animals were sacrificed. Liver samples were taken from several lobes and frozen in liquid nitrogen. Serum samples were also collected.

### Liver Function Tests

Routine liver function blood tests, including hyaluronan, type IV collagenase, γGT and transaminases were performed on an automated analyzer.

### Fibrosis Assessment

Fibrosis assessment was done on formalin-fixed, paraffin-embedded sections stained with HE. Pictures were taken, using an image analysis system. All samples from a series of experiments were stained simultaneously. Liver fibrosis deposition was judged by senior pathologists and graded with the METAVIR scale, which grades fibrosis from F0 (no fibrosis) to F4 (cirrhosis). The METAVIR scale is a widely used scale that has excellent inter-observer reliability. Table 3 and Table 4 show the results.

### Statistical Analysis

Statistical analysis in Tables 3 and 4 was performed by WILCOXON in other tables and Figures by ANOVA using the SPSS11.0 software.

### Results

As expected, CCl4 treatment for 5 weeks induced significant liver damage and fibrogenesis, including mononuclear cell infiltration, cellular ballooning, fatty degeneration and hepatic lobules separated by fibs. Addition of Fibroscutum decreased the pathologic changes to slight fatty degeneration of liver cells and no significant fibrogenesis (Table 3).

TABLE 3
Pathological changes of rat liver after 5 weeks of CCL4 treatment and 3
weeks of Fibroscutum treatment

| | | Pathologic morphology of rat liver slices | | | | | | | |
|---|---|---|---|---|---|---|---|---|---|
| | | Cell damage | | | | Fibrosis | | | |
| Group | No. of rat | − | + | ++ | +++ | F0 | F1 | F2 and 3 | F4 |
| control | 7 | 7 | | | | 7 | | | |
| CCL4 | 7 | | | 3 | 4 | | 3 | 4 | |
| Fibroscutum | 7 | | 1 | 6 | | | 5 | 2 | |

| | | |
|---|---|---|
| CCL4 v Control | P < 0.01 | P < 0.01 |
| CCL4 v Fibroscutum | P < 0.05 | P > .05 |

Compared with controls, eight weeks of CCL4 treatment induced severe fibrosis with the formation of nodules that lack a central vein. As shown in FIG. 1A, the normal structure of hepatic lobules disappeared. As shown in FIG. 1B, the addition of Fibroscutum decreased the pathologic changes to slight fibrogenesis. Table 4 shows the statistic results.

TABLE 4
Pathological changes of rat liver after 8 weeks of CCL4 treatment
and 6 weeks of Fibroscutum treatment

| | | Pathologic morphology of rat liver slices | | | | | | | |
|---|---|---|---|---|---|---|---|---|---|
| | | Cell damage | | | | Fibrosis | | | |
| Group | No. of rat | − | + | ++ | +++ | F0 | F1 | F2 and 3 | F4 |
| control | 7 | 7 | | | | 7 | | | |
| CCL4 | 7 | | 1 | 2 | 4 | | | 1 | 6 |
| Fibroscutum | 7 | | 4 | 3 | | | 3 | 3 | 1 |

| | | |
|---|---|---|
| CCL4 v Control | P < 0.01 | P < 0.01 |
| CCL4 v Fibroscutum | P < 0.05 | P < 0.01 |

Treatment of Fibroscutum on the CCL4-treated rats resulted in a reduction in fibrosis. The results were the most significant in the group of rats following 8 weeks of CCL4 treatment and 6 weeks of Fibroscutum treatment, as compared with CCL4 treatment alone (p<0.0001 by WILCOXON). Biochemical tests of liver function are presented in Table 5 and Table 6.

TABLE 5
Liver function tests of rats after 5 weeks of CCL4 treatment and 3 weeks of
Fibroscutum treatment

| | | HA | cIV | ALT | AST | γ-GT |
|---|---|---|---|---|---|---|
| Group | N | ng/ml | ng/ml | u/ml | u/m | u/ml |
| control | 7 | 41.91 ± 18.77 | 18.87 ± 10.29 | 34.23 ± 10.7 | 98.86 ± 11.94 | 13.00 ± 6.71 |
| CCL4 | 7 | 328.69 ± 92.81 | 206.39 ± 70.28 | 135.31 ± 24.23 | 158.29 ± 17.37 | 45.00 ± 22.61 |
| Fibroscutum | 7 | 66.31 ± 21.99 | 74.43 ± 19.10 | 42.5 ± 12.29 | 118.00 ± 8.17 | 28.00 ± 19.40 |

| | | | | | |
|---|---|---|---|---|---|
| CCL4 v Control | P < 0.01 | P < 0.01 | P < 0.01 | P < 0.01 | P < 0.01 |
| CCL4 v | P < 0.01 | P < 0.01 | P < 0.01 | P < 0.01 | P > .05 |
| Fibroscutum | | | | | |

TABLE 6
Liver function tests of rats after 8 weeks of CCL4 treatment and 6 weeks of
Fibroscutum treatment

| | | HA | cIV | ALT | AST | γ-GT |
|---|---|---|---|---|---|---|
| Group | N | ng/ml | ng/ml | u/ml | u/m | u/ml |
| control | 7 | 35.92 ± 12.23 | 46.49 ± 15.84 | 32.03 ± 9.05 | 100.71 ± 19.99 | 15.14 ± 11.71 |
| CCL4 | 7 | 130.39 ± 52.27 | 135.98 ± 28.45 | 132.97 ± 32.05 | 166.57 ± 28.79 | 74.43 ± 35.50 |
| Fibroscutum | 7 | 58.19 ± 20.18 | 57.03 ± 20.60 | 47.10 ± 10.41 | 139.57 ± 14.42 | 2.14 ± 21.08 |

| | | | | | |
|---|---|---|---|---|---|
| CCL4 v Control | P < 0.01 | P < 0.01 | P < 0.01 | P < 0.01 | P < 0.01 |
| CCL4 v | P < 0.01 | P < 0.01 | P < 0.01 | P < 0.01 | P > .05 |
| Fibroscutum | | | | | |

The results in Tables 5 and 6 are expressed as means±1 SD.

The difference between the serum biochemical tests of liver function between rats of CCL4 treatment alone and CCL4 plus Fibroscutum is statistically significant except for r-GT.

In Table 5 and Table 6, N=number of animals used.

During liver fibrogenesis, since there is extensive deposition of fibrous tissue, serum levels of the constituents of extracellular matrix such as hyaluronic acid (HA), collagen VI (cIV) and many of their breakdown products will increase as a result of remodeling and recurrent scarring.

Serum alanine and aspartate aminotransferase (ALT and AST) levels do not correlate well with fibrosis. However, patients with documented, persistently normal ALT levels usually have mild degrees of hepatitis and either no or mild stages of fibrosis. collagen VI (cIV) and many of their breakdown products will increase as a result of remodeling and recurrent scarring.

Serum alanine and aspartate aminotransferase (ALT and AST) levels do not correlate well with fibrosis. However, patients with documented, persistently normal ALT levels usually have mild degrees of hepatitis and either no or mild stages of fibrosis.

### Example 2

### Inhibition of Fibrogenesis by Fibroscutum Through Suppressing Activation of HSC Cells

To elucidate the action of Fibroscutum in fibrogenesis, the effect of Fibroscutum on activation of HSC cells stimulated by TGF-β 1 was measured. HSC activation has been examined and is an essential process of liver fibrogenesis. It presents as proliferation of HSC and fibroblast, and remodeling of extra cellular matrix, including, degradation of collagen IV in basement membrane and deposition of an excess of extracellular matrix components, such as collagen I and collagen III, and change of expression of MMPs and TIMPs in mRNA level. TGF-β 1 is one of the most important activation factors in the process.

### Material and Methods

### Cells

An established Human HSC cell line was used, named LX-2. These cells have been extensively characterized and exhibit many similarities with primary cultures of HSC.

### Cell Proliferation Assay

LX-2 cells (2000/well) were seeded in 96-well microplate for 24 hr, the culture medium was replaced with DMEM supplemented with 0.5% FBS and allow cells to be starved for 48 hr. After starvation, the medium was replaced with 2% serum supplemented DMEM medium. Then the reagents were added to treat cells and LX-2 cells were exposed to Fibroscutum, either alone, or in the presence of 2 ng/ml TGF.beta.1 for 48 hours. Cell number was evaluated through the measurement of the reduction of the dye 3-(4,5-dimethylthiazol-2yl)-2,5 diphenyltetrazolium (MTT).

### Expression of Matrix Metalloproteinases-2 (MMP-2) and Tissue Inhibitor of Matrix Metalloproteinases-1 (TIMP-1) mRNA Levels

MMP-2 is the enzyme which can degrade Collagen IV; and TIMP-1 increases fibrosis deposition by inhibiting its degradation by matrix metalloproteinases. The effect of Fibroscutum on MMP-2 and TIMP-1 mRNA expression by cultured LX-2 cells was measured. 2×10⁵ LX-2 cells were seeded in 6-well cell culture plate for 24 hours. Then the culture medium was replaced with DMEM supplemented with 0.5% FBS to allow cells to be starved for 24 hours. After starvation, the medium was replaced with 2% serum supplemented DMEM medium. Then the reagents were added to treat cells and LX-2 cells were exposed to Fibroscutum, either alone, or in the presence of 2 ng/ml TGF.beta.1 for 24 hours. Finally, cells were harvested and lysised using TRIZOL reagent (GIBCOL, MD, USA). The total RNA was then extracted according to manufacturer's protocol. RT-PCR was carried out. The gene expression levels of MMP-2 and TIPM-1 were normalized by a housekeeping gene, glyceraldehydes-3-phosphate dehydrogenase (GADPH).

### Expression of Collagen Type I and III Protein Levels

Deposition of an excess of extracellular matrix components, such as Collelagen I and III is a pathologic process during fibrogenesis. The effect of Fibroscutum on MMP-2 and TIMP-1 mRNA expression by cultured LX-2 cells was measured. 2×10⁵ LX-2 cells were seeded in 6-well cell culture plate for 24 hours. Then the culture medium was replaced with DMEM supplemented with 0.5% FBS and cells were starved for 24 hours. After starvation, the medium was replaced with 2% serum supplemented DMEM medium, The reagents were then added to treat cells and LX-2 cells were exposed to Fibroscutum, either alone, or in the presence of 2 ng/ml TGF.beta.1 for 48 hours. The culture medium was harvested and Western Blotting was carried out to evaluate the effect of Fibroscutum on collagen I and III levels of LX-2 cells treated with or without TGF-β 1.

### Results

### Proliferation

FIG. 2 is a graph illustrating the effect of Fibroscutum on hepatic stellate cell proliferation induced by TGF.beta.1. LX-2 cells (Human HSC cell line) were exposed to Fibroscutum, either alone, or in the presence of 2 ng/ml TGF.beta.1 for 48 hours. Cell number was evaluated through the measurement of the reduction of the dye 3-(4,5-dimethylthiazol-2yl)-2,5 diphenyltetrazolium (MTT). In FIG. 2, C represents control; F represents Fibroscutum (26 μg/ml); T represents TGF.beta.1 (2 ng/ml); T+FS represents TGF.beta.1 (2 ng/ml) plus Fibroscutum (26 μg/ml). (* means control v TGF.beta.1 p<0.05; ++ means TGF.beta.1 v TGF.beta.1 plus Fibroscutum p<0.01. N=4.)

As shown in FIG. 2, TGF-β 1 increased LX-2 cell proliferation. The mitogenic effect of TGF-β 1 (2 ng/ml) on LX-2 was abolished by simultaneous addition of Fibroscutum. Fibroscutum alone also had slight effect, although the slight effect might have been caused by the inhibition of the small amount of intrinsic TGF-β 1 produced by LX-2 cell.

### MMP-2 and TIMP-1

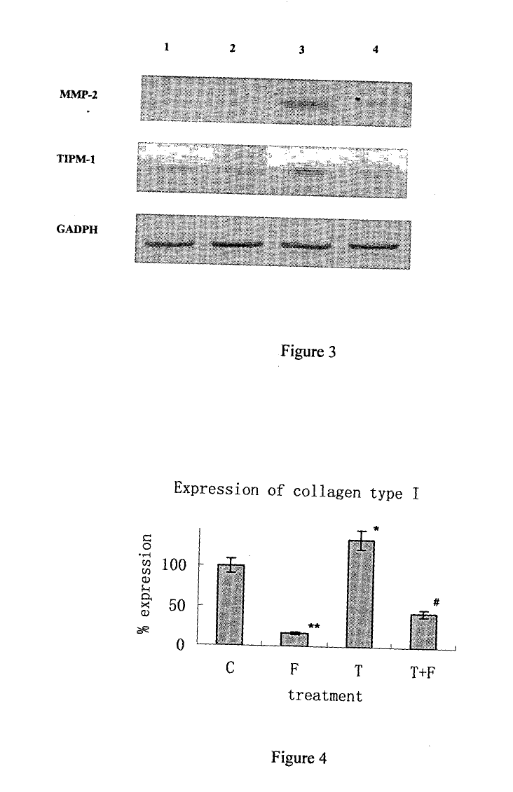

As shown on FIG. 3, TGF-β 1(2 ng/ml) increased the mRNA expression levels of MMP-2 and TIMP-1 in LX-2 cells, whereas Fibroscutum suppressed the mRNA expression levels of MMP-2 and TIMP-1 in LX-2 cells. Note, RT-PCR was carried out to detect the effect of Fibroscutum on MMP-2 and TIMP-1 mRNA levels of LX-2 cells stimulated with TGF.beta.1. GADPH was used as a control. In FIG. 3, 1 represents control; 2 represents Fibroscutum (26 μg/ml); 3 represents TGF.beta.1 (2 ng/ml); 4 represents TGF.beta.1 (2 ng/ml) plus Fibroscutum (26 μg/ml).

### Collagen Type I and III

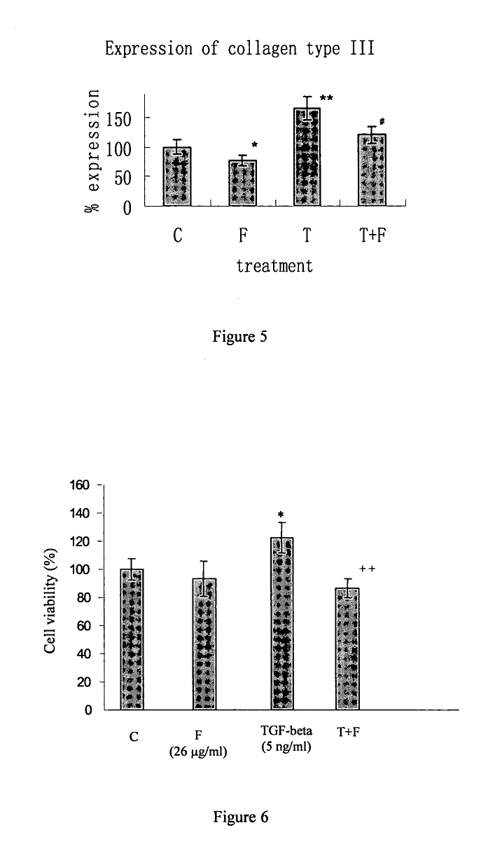

FIGS. 4 and 5 show that TGF-β 1 (2 ng/ml) increased the protein levels of Collagen Type I and III in culture medium of LX-2 cells. Fibroscutum suppressed the effects of TGF-β 1. Fibroscutum treatment alone also caused a significant decrease in the protein levels of Collagen Type I and III in LX-2 cells. This may be due to the effect of Fibroscutum on the small amount of intrinsic TGF-β 1 produced by LX-2 cells. Note in FIG. 4 Western Blotting was carried out to evaluate the effect of Fibroscutum on collagen I protein levels of LX-2 cells stimulated with TGF.beta.1. C represents control; F represents Fibroscutum (26 μg/ml); T represents TGF.beta.1 (2 ng/ml); T+FS represents TGF.beta.1 (2 ng/ml) plus Fibroscutum (26 μg/ml). (** means control v Fibroscutum p<0.01; * means control v TGF.beta.1 p<0.05; # means TGF.beta.1 v Fibroscutum plus TGF.beta.1 p<0.05. N=4). Also in FIG. 5, Western Blotting was carried out to evaluate the effect of Fibroscutum on collagen III protein levels of LX-2 cells stimulated with TGF.beta.1. C represents control; F represents Fibroscutum (26 μg/ml); T represents TGF.beta.1 (2 ng/ml); T+FS represents TGF.beta.1 (2 ng/ml) plus Fibroscutum (26 μg/ml.). (** means control v Fibroscutum p<0.01; * means control v TGF.beta.1 p<0.05; # means TGF.beta.1 v Fibroscutum plus TGF.beta.1 p<0.05. N=4).

### Example 3

### The Effect of Fibroscutum on Proliferation of NIH 3T3 Cells Stimulated by TGF.beta.1

Proliferation of fibroblasts is also involved in fibrogenesis. The effect of Fibroscutum on fibroblast proliferation was therefore examined.

### Cells

NIH 3T3 fibroblasts were bought from ATCC.

### Proliferation

NIH 3T3 fibroblasts (2000/well) were seeded in 96-well microplate for 24 hours. The culture medium was replaced with DMEM supplemented with 0.5% FBS and the cells were starved for 48 hours. After starvation, the medium was replaced with 2% serum supplemented DMEM medium. The reagents were then added to treat cells; and NIH 3T3 cells were exposed to Fibroscutum, either alone, or in the presence of 5 ng/ml TGF.beta.1 for 48 hours. Cell number was evaluated through the measurement of the reduction of the dye 3-(4,5-dimethylthiazol-2yl)-2,5 diphenyltetrazolium (MTT).

### Results

FIG. 6 shows that TGF-β 1 increased the proliferation of NIH-3T3 fibroblast cells. However, the mitogenic effect of TGF-β 1 (5 ng/ml) on fibroblasts was suppressed by simultaneous addition of Fibroscutum. In FIG. 6, NIH 3T3 Cells were exposed to Fibroscutum, either alone, or in the presence of 2 ng/ml TGF.beta.1 for 48 hours. Cell number was evaluated through the measurement of the reduction of the dye 3-(4,5-dimethylthiazol-2yl)-2,5 diphenyltetrazolium (MTT). C represents control; F represents Fibroscutum (26 μg/ml); T represents TGF.beta.1 (2 ng/ml); T+FS represents TGF.beta.1 (2 ng/ml) plus Fibroscutum (26 μg/ml.). (* means control v TGF.beta.1 p<0.05; ++ means TGF.beta.1 v Fibroscutum plus TGF.beta.1 p<0.05. N=4.)

### Example 4

### Production Procedure of Fibroscutum

The following presents one method for producing Fibroscutum:

i) Homogenization

Extraction buffer is added to the liver (30/100 W/V) and homogenized at top-speed for 5 minutes. The procedure is repeated three times, each with a 2-minute pause to make sure the liver tissue and cells are broken.

ii) Mixing

The homogenized solution is mixed at 60 rpm for one hour at room temperature for maximum extraction yield.

iii) Heating

The homogenized sample is added to the preheated (95° C.) water bath and is kept at 95° C. for 15 minutes with low-speed stirring. The temperature must not be higher than 95° C. Otherwise, the bioactivity will be reduced. However, if the temperature is lower than 95° C., the purity of the product will be affected.

iv) Filtration and Cooling

The sample is ground and filtered after heating. The filtered solution was then water-cooled to room temperature.

v) Purification

v-a) Packing of Column

Wash, clean and install column for use. Gently shake DEAE Sepharose gel to make it into an even slurry. Dilute gel suspension with 2 column load of PB buffer. Pour well-mixed gel suspension gently down the wall of column using a glass rod. Pour all gel suspension in one operation. Fill the top of column reservoir with PB buffer.

v-b) DEAE Sepharose Treatment

i) Wash column with one column load of NaCl. Then wash column again with 3 column load of distilled water.

ii) Then wash column again with 2 column load of NaOH. Sterilize column for 6 hours at room temperature. Wash column again with distilled water until column pH is 7.0.

iii) Maintain column pH at 7.6 by adjusting the volume of PB buffer.

v-c) Eluting with Solution A

After column is packed, solution A is applied at a flow rate of 4000 ml/hour and the elution is examined at OD₂₈₀. When OD is less than 0.5, the eluting process is to be stopped. The eluate is then removed as waste.

v-d) Eluting with Solution B

When OD is less than 0.5, solution B is to be used in place of solution A and the flow rate is to be set at 2000 ml/hour. The elution is examined by OD₂₈₀. When the OD starts to increase, the eluate is to be collected until the OD₂₈₀ reading is less than 0.5.

v-e) Desalting

Desalting is performed using ultra-filtration membrane and the desalted product is the Fibroscutum bulk.

v-f) Bulk Test

vi-a) Protein Concentration

The protein concentration is determined using the Lowry method. The concentration is adjusted to 15 μg/ml using injection water to produce the final product.

vi-b) Adjusting pH

pH is measured using a pH meter to ensure that the pH of the final product is 7.0-7.8.

vi-c) Chloride Test

The AgNO₃ quantitative determination method is used to test the concentration of chloride. The required criterion is that the concentration of chloride be less than 1.0 mmol/L.

vii) Sterilization

The sample is sterilized using a 0.2μ filter. The sample is filtered twice to achieve the desired level of sterilization.

viii) Bottling

The sample is then ready for bottling.

ix) Storage

The bottled sample is to be stored at 4° C.

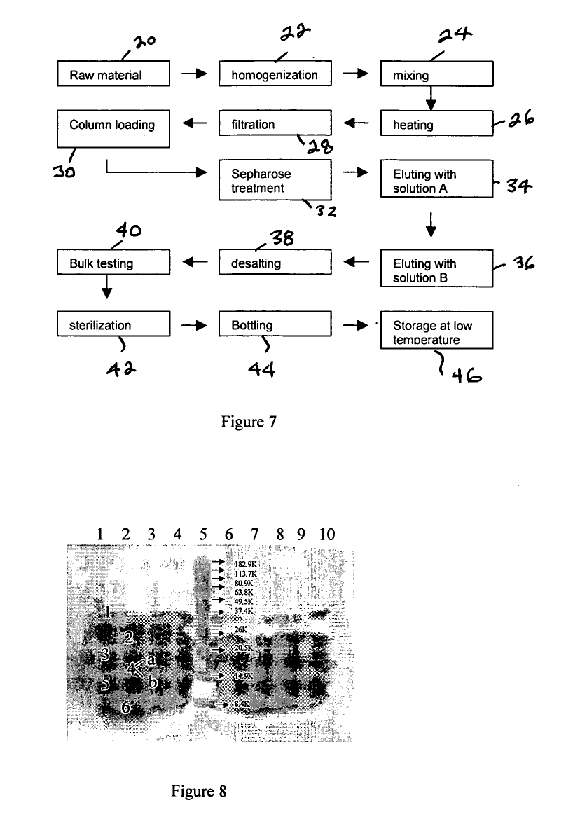

FIG. 7 shows a flow chart describing the Fibroscutum production procedure. Here raw material 20 goes through a homogenization process 22 followed by mixing 24, a heating step 26, a filtration step 28 and a column-loading step 30. The result is provided for Sepharose treatment 32 followed by eluding with solution A 34. This in turn is followed by elution with solution B at 36, desalting at 38 and bulk testing at 40. The bulk-tested constituents are sterilized as illustrated at 42, followed by bottling as illustrated at 44 and storage at low temperature as illustrated at 46.

Note that in FIG. 8, Fibroscutum was concentrated to 1.58 ug/ul and loaded to lane 1-4 and 6-10. Lane 5 was protein markers. The mixture was separated by 17% SDS PAGE and the gel was stained by Coomassie Blue. Six major bands were found in Fibroscutum. The estimated molecular weights of these major bands are 39.81, 24.92, 20.65, 14.17, 12.06, 10.20 and 7.34 KDa.

In summary, the above data demonstrates that Fibroscutum inhibited liver fibrogenesis through suppressing HSC activation induced by TGF-β1, including suppressing TGF-β1 induced cell proliferation, collagen I and III production, and MMP-2 and TIMP-1 expression. Fibroscutum also inhibited proliferation of fibroblast induced by TGF-β.

While the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

1. A method for inhibiting hepatic fibrogenesis, which method comprises administering an effective amount of an antagonist of hepatic stellate cell activation and fibroblast cell activation to a patient in need of such treatment.

2. The method according to claim 1, wherein the antagonist prevents the development of liver fibrosis in the course of viral hepatitis and/or induced liver damage.

3. The method according to claim 1, wherein the antagonist prevents the development of liver fibrosis in the course of chronic hepatitis and/or induced liver damage.

4. The method according to claim 1, wherein the antagonist is an antagonist of fibrotic conditions related to hepatic stellate cell activation and/or and fibroblast cell activation.

5. The method according to claim 1, wherein the antagonist is Fibroscutum, a mixture of natural peptides extracted from the liver tissue of suckling pig, containing at least 6 peptides of relative abundance of 10-27% and molecular weight of 7-40 kD.

6. A method of suppressing growth of tumor cells, including hepatocellular carcinoma, using Fibroscutum.

7. A method for preventing or reducing fibrotic conditions using Fibroscutum.

8. The method of claims 6 and 7, wherein Fibroscutum is a peptide mixture extracted from the liver tissue of suckling pig, containing at least 6 peptides of relative abundance of 10-27% and molecular weight of 7-40 kD.

9. A method for the preparation of Fibroscutum, comprising the steps of:
homogenizing livers from piglets;
mixing the homogenized livers at 60 rpm for 1 hour at room temperature;
heating the mixed homogenized livers at 95° C.;
filtering the heated mixture;
cooling the filtered mixture to provide a sample;
purifying the sample with DEAE Sepharose in a fast-flow process; and,
sterilizing the purified sample by filtration with a 0.2 u filter.
